(19) United States
(12) Patent Application Publication
(10) Pub. No.: US 2025/0064696 A1
(43) Pub. Date: Feb. 27, 2025

(54) Oral Care Compositions and Methods for the Same

(71) Applicant: Colgate-Palmolive Company, New York, NY (US)

(72) Inventors: Yuwei CHEN, Guangzhou (CN); Shaopeng XU, Guangzhou (CN); Xu Qiang GAO, Guangzhou (CN)

(73) Assignee: Colgate-Palmolive Company, New York, NY (US)

(21) Appl. No.: 18/724,716

(22) PCT Filed: Dec. 21, 2022
(86) PCT No.: PCT/US2022/053666

§ 371 Date: Jun. 27, 2024

(30) Foreign Application Priority Data
Jan. 4, 2022 (CN) 202210003631.3

(51) Int. Cl. A61K 8/34; A61Q 11/00

(52) U.S. Cl. CPC A61K 8/347; A61Q 11/00; A61K 2800/522

(57) ABSTRACT

Oral care compositions having antioxidant efficacy are disclosed. The oral care composition may include an orally acceptable vehicle and one or more phenolic compounds. The phenolic compounds may be capable of or configured to provide or improve antioxidant efficacy of the oral care composition. Methods for preparing the oral care compositions, methods for treating, preventing, or inhibiting one or more conditions of an oral cavity resulting from oxidative damage, and methods for scavenging one or more free radicals in an oral cavity are also disclosed.

### CROSS-REFERENCE TO RELATED APPLICATIONS

This application claims priority to Chinese Patent Application No. 202210003631.3, filed on Jan. 4, 2022, the contents of which are incorporated herein by reference to the extent consistent with the present disclosure.

### BACKGROUND

Oxidative damage of the oral cavity and soft tissue thereof may often result in inflammation, periodontal disease, and other health related conditions, such as cardiovascular disease or increased susceptibility to cancer. Oxidative damage of the oral cavity and the soft tissue thereof may originate from various sources or have many causes. For example, the oxidative damage may often result from exposure to chemicals, such as from tobacco products or alcohol. In other examples, the oxidative damage may result from natural processes of the body. The commonality between many of the sources of oxidative damage is free radicals and/or other reactive oxygen species.

While antioxidants are well known for addressing the formation or presence of free radicals, effective administration of the antioxidants to the oral cavity and the soft tissue thereof is limited. For example, antioxidants are conventionally ingested in food or as nutritional supplements. As such, the distribution of the antioxidants to particular or targeted parts of the body, such as the oral cavity, is difficult to regulate, thereby limiting the antioxidant effect in the oral cavity.

What is needed, then, are oral care compositions and methods to prevent, reduce, or otherwise inhibit the formation of free radicals in the oral cavity.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing an oral care composition including an orally acceptable vehicle and one or more phenolic compounds. The phenolic compounds may be configured to provide or improve antioxidant efficacy of the oral care composition.

In at least one implementation, the one or more phenolic compounds may include one or more of hydroquinone, guaiacol, eugenol, gallic acid, any derivative thereof, or a combination thereof.

In at least one implementation, the one or more phenolic compounds may include eugenol or a derivative thereof.

In at least one implementation, the one or more phenolic compounds may include eugenol.

In at least one implementation, the one or more phenolic compounds may include a derivative of eugenol.

In at least one implementation, the derivative of eugenol may include one or more of eugenyl acetate, eugenyl formate, eugenyl benzoate, eugenol methyl ether, eugenol amyl ether, isoeugenol, dihydroeugenol, acetyl eugenol, methyleugenol, or combinations thereof.

In at least one implementation, the one or more phenolic compounds may be present in the oral care composition in an amount effective to treat, prevent, or inhibit one or more conditions of the oral cavity caused by or resulting from oxidative damage.

In at least one implementation, the one or more phenolic compounds may be present in an amount greater than or equal to 0.005%, greater than or equal to 0.006%, greater than or equal to 0.007%, greater than or equal to 0.008%, greater than or equal to 0.009%, or greater than or equal to 0.01%%, based on the total weight of the oral care composition.

In at least one implementation, the one or more phenolic compounds may be present in an amount of from greater than or equal to about 0.005% to about 0.025%, more preferably, from greater than or equal to about 0.008% to about 0.025%.

In at least one implementation, the orally acceptable vehicle may include one or more thickening agents, one or more humectants, one or more solvents, one or more pH modifying agents, one or more flavorants, one or more preservatives, one or more additional antioxidants, one or more sweeteners, one or more fluoride ion sources, one or more antimicrobial agent, one or more tartar control agents, or combinations thereof.

The foregoing and/or other aspects and utilities embodied in the present disclosure may be achieved by providing a method for preparing any one or more of the oral care compositions disclosed herein. The method may include contacting the orally acceptable vehicle and the one or more phenolic compounds with one another.

The foregoing and/or other aspects and utilities embodied in the present disclosure may be achieved by providing a method for treating, preventing, or inhibiting one or more conditions of an oral cavity resulting from oxidative damage. The method may include contacting any of the oral care compositions disclosed herein with the oral cavity and/or a surface thereof.

The foregoing and/or other aspects and utilities embodied in the present disclosure may be achieved by providing a method for scavenging one or more free radicals in an oral cavity. The method may include contacting any of the oral care compositions disclosed herein with the oral cavity

In at least one implementation, the method may include measuring a decrease in free radicals in the oral cavity.

In at least one implementation, the method may include diagnosing the presence of an oral condition resulting from oxidative damage in the oral cavity.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that oral care compositions including phenolic compounds, such as eugenol or a derivative thereof, provided a statistically significant or unexpected increase in antioxidant activity or efficacy.

Particularly, it was surprisingly discovered that oral care compositions including eugenol or a derivative thereof in an amount of at least 0.008 wt % provided a statistically significant or unexpected increase in antioxidant activity or efficacy. It was further surprisingly and unexpectedly discovered that a concentration of about 0.008 wt % was the lowest dosage that provided the best performance or best antioxidant activity, as increasing amounts of eugenol provided no statistically significant benefit with respect to the antioxidant efficacy or capacity, free radical scavenging ability, and/or antioxidant capacity of the oral care composition.

Compositions disclosed herein may be or include an oral care product or an oral care composition thereof. For example, the composition may be an oral care product including the oral care composition and/or one or more additional ingredients/components. In another example, the composition may be the oral care composition of the oral care product. As used herein, the expression “oral care product” may refer to the final form which is sold to a consumer or administered to a user (e.g., patient). The oral care product may be a product that includes one or more chemical compounds or chemical compositions that may be applied to an oral cavity or a surface (e.g., soft tissue) thereof to therapeutically or non-therapeutically treat a condition (e.g., reduce free radical or prevent oxidative damage), deliver a benefit agent, improve the health of the user's oral cavity (e.g., vestibule, lips, jaws, palate, teeth, tongue, etc.), or a combination thereof.

The oral care product or the oral care composition thereof may be a liquid, a fluid, a gel, or a paste. Illustrative oral care products or compositions of the present disclosure may be or include, but are not limited to, a toothpaste (dentifrice), an oral rinse, a mouth rinse, a denture cleaner, a saliva substitute, a mouthwash, an oral balm, a serum (e.g., concentrated product), a serum pen, an oral gel, such as a leave-on gel, or any other oral care product intended to contact the oral cavity and/or one or more surfaces of the oral cavity. As used herein, the terms or expressions “mouthwash,” “mouth rinse,” “oral rinse,” or the like, may refer to a liquid that contacts surfaces of the oral cavity or mouth passively or actively. In a preferred implementation, the oral care product or the composition thereof is a mouthwash.

The compositions disclosed herein may be or include oral care compositions including an orally acceptable vehicle or carrier and one or more phenolic compounds. As further described herein, the one or more phenolic compounds may be an antioxidant capable of or configured to provide or improve antioxidant efficacy of the oral care composition. As used herein, the term or expression “antioxidant” may refer to a substance that may scavenge free radicals and/or other reactive oxygen species, prevent the formation of free radicals and/or other reactive oxygen species, convert the free radicals and/or the other reactive oxygen species to a relatively less reactive species, or combinations thereof. As used herein, the term or expression “antioxidant efficacy” may refer to the ability or an acceptable measure of the ability of a substance to scavenge free radicals and/or other reactive oxygen species, prevent the formation of free radicals and/or other reactive oxygen species, convert the free radicals and/or the other reactive oxygen species to a relatively less reactive species, or combinations thereof.

The one or more phenolic compounds may be or include, but are not limited to, hydroquinone, guaiacol, eugenol, gallic acid, any derivatives thereof, or any combination thereof. In a preferred implementation, the one or more phenolic compounds include eugenol and/or a derivative thereof. Eugenol or 4-Allyl-2-methoxyphenol is an essential oil extracted from clove oil, nutmeg, cinnamon, basil and bay leaves and has a clear pale yellow color. Eugenol has low solubility in water and is well soluble in organic solvents. It also has a clove aroma as a fragrance. As such, at relatively high concentration, eugenol may present a relatively unpleasant aroma and/or flavor. Eugenol is a major component of essential oils extracted from cloves. Derivatives of eugenol may be or include, but are not limited to, esters, ether, acetal derivatives of eugenol, such as eugenyl acetate, eugenyl formate, eugenyl benzoate, eugenol methyl ether, and eugenol amyl ether, isoeugenol, dihydroeugenol, acetyl eugenol, methyleugenol, or combinations thereof. It should be appreciated that the derivatives of eugenol may generally have or exhibit relatively decreased or less flavor or odor as compared to eugenol, thereby providing a milder aroma or taste. As such, utilizing a derivative of eugenol may provide the antioxidant functionality of eugenol while providing a relatively more pleasant flavor and/or aroma. As further described herein, the phenolic compound, namely, eugenol and/or a derivative thereof, may be capable of or configured to therapeutically or non-therapeutically treat, prevent, or otherwise inhibit one or more conditions of the oral cavity caused by or resulting from oxidative damage.

The one or more phenolic compounds (e.g., eugenol) may be present in the oral care composition in an amount effective to treat, prevent, or otherwise inhibit one or more conditions of the oral cavity caused by or resulting from oxidative damage. The one or more phenolic compounds may also be present in the oral care composition in an amount effective to act as an antioxidant to thereby scavenge, reduce, react, prevent the formation of, or otherwise interact with one or more free radicals and/or other reactive oxygen species in the oral cavity and/or on a surface thereof (e.g., soft tissue). The one or more phenolic compounds may be present in the oral care composition in an amount greater than or equal to 0.005%, greater than or equal to 0.006%, greater than or equal to 0.007%, greater than or equal to 0.008%, greater than or equal to 0.009%, or greater than or equal to 0.01%%, based on the total weight of the oral care composition. The one or more phenolic compounds may also be present in the oral care composition in an amount less than or equal to about 1%, less than or equal to about 0.1%, less than or equal to 0.05%, less than or equal to about 0.025%, or less than or equal to about 0.015%, based on the total weight of the oral care composition. In a preferred implementation, the one or more phenolic compounds includes eugenol and/or one or more derivatives thereof, and the eugenol and the derivatives thereof may be present in an amount of from greater than or equal to about 0.005% to about 0.025%, more preferably from greater than or equal to about 0.008% to about 0.025%.

As used herein, the expression “orally acceptable vehicle” or “carrier” may refer to a suitable vehicle, ingredient, or combination of ingredients, which may be utilized to dissolve, disperse, suspend, hold, mobilize, or otherwise contain the one or more phenolic compounds. In a preferred implementation, the composition includes eugenol and/or one or more derivatives thereof dispersed or otherwise contained in the orally acceptable vehicle or carrier. The orally acceptable vehicle may include one or more thickeners or thickening agents, one or more humectants, one or more solvents, one or more pH modifying agents, one or more flavorants or flavoring agents, one or more preservatives (e.g., natural benzyl alcohol), one or more additional antioxidants, one or more sweeteners, one or more additional ingredients, one or more fluoride ion sources, one or more antimicrobial agent and/or preservatives, one or more tartar control agents, or a combination thereof.

The one or more thickeners or thickening agents may be or include one or more polymers capable of or configured to modify (i.e., increase or decrease) the viscosity of the oral care composition. The one or more thickeners or the polymers thereof may be or include, but are not limited to, one or more nonionic thickening polymers, one or more anionic thickening polymers or gelling agents, or combinations thereof. The one or more polymers may be or include water-dispersible or water-soluble hydrophilic colloids.

The one or more polymers of the thickening agents may be or include polysaccharides. The one or more polymers or polysaccharides thereof may be unmodified, as isolated from their source materials, or may be modified as is well known in the polymer art, such as by acetylation, hydroxyalkylation, carboxyalkylation, hydroxyalkylation carboxyalkylation, cationic substitution, or a combination thereof. The one or more polymers of the thickening agents may be or include natural and/or modified natural polymers and gums. Illustrative polysaccharides may be or include, but are not limited to, cellulose derivatives (e.g., carboxymethyl cellulose), gums or polysaccharide gums, or a combination thereof. Illustrative gums or polysaccharide gums may be or include, but are not limited to, xanthan gum, carrageenan gum, guar gum, succinoglucan gum, welan gum, gum Arabic, tragacanth gum, locust bean gum, or the like, or a combination thereof.

The anionic polymeric thickeners of the thickening agents may include polyacrylates, such as acrylate-alkyl acrylate copolymers, preferably those selected from carbomers or carbopols (CARBOPOL®, commercially available from Lubrizol Corp. of Wickliffe, OH). Carbomers are homopolymers of acrylic acid crosslinked with an allyl ether of pentaerythritol, sucrose, or propylene. Illustrative acrylate copolymers and/or acrylate-alkyl acrylate copolymers may be or include, but are not limited to, CARBOPOL® 1382, CARBOPOL® 981, CARBOPOL® 5984, AQUA SF-1, or the like, or combinations thereof, each of which are commercially available from Lubrizol Corp., or the like, or combinations thereof. Illustrative acrylate copolymers and/or acrylate-alkyl acrylate copolymers may be or include, but are not limited to, those having INCI name acrylates/C10-30 alkyl acrylate crosspolymer, such as CARBOPOL® 1382, CARBOPOL® ETD 2020, CARBOPOL® Ultrez 21, PEMULEN TR1, PEMULEN TR2, or the like, or combinations thereof, each of which are commercially available from Lubrizol Corp. The acrylates/C10-30 alkyl acrylate crosspolymers are copolymers of C10-30 alkyl acrylates and one or more monomers of acrylic acid, methacrylic acid or their simple esters thereof crosslinked with an allyl ether of sucrose or pentaerythritol. In a preferred implementation, the one or more thickening agents include a polyacrylate thickener, more preferably, an acrylates/C10-30 alkyl acrylate crosspolymer, even more preferably CARBOPOL® ETD 2020.

It should be appreciated that the acrylates/C10-30 alkyl acrylate crosspolymer may be capable of or configured to facilitate or increase viscosity and/or thickening without heating. The acrylates/C10-30 alkyl acrylate crosspolymer may also be capable of or configured to provide a smooth, sheer thinning texture similar to conventional lip balm, thereby providing a smooth and soothing feeling to soft tissue (e.g., gums, cheeks, tongue, etc.). The acrylates/C10-30 alkyl acrylate crosspolymer may also be capable of or configured to provide a protective barrier on soft tissue or surfaces thereof to prevent, deflect, inhibit microbes (e.g., bacteria) from attaching to the soft tissue. For example, the acrylates/C10-30 alkyl acrylate crosspolymer may promote, provide, or improve barrier integrity as a mucoadhesive polymer. The acrylates/C10-30 alkyl acrylate crosspolymer is an anionic, mucoadhesive polymer that increases the substantivity and film-forming behavior of formulations. A mucoadhesive film of hydrated Carbopol may act as a protective barrier, thereby shielding soft tissue and/or surfaces thereof from bacterial attachment, irritation, environmental stressors (e.g., air pollution, LPS, cigarette smoke, etc.), or combinations thereof.

The one or more thickening agents may be present in an amount effective to sufficiently increase the viscosity of the oral care composition. For example, any one or more of the thickening agents may be present in an amount of from about 0.01 wt % to about 40 wt %, based on the total weight of the composition. In another example, any one or more of the thickening agents may be present in an amount of from about 0.05 wt % to about 10 wt %, based on the total weight of the composition. For example, any one or more of the thickening agents may be present in an amount of from about 0.01 wt % to about 20 wt %, preferably, about 0.05 wt % to about 15 wt %, more preferably about 0.05 wt % to about 10 wt %, even more preferably about 0.1 wt % to about 2 wt %, or about 1 wt %, based on the total weight of the composition. In a preferred implementation, the one or more thickening agents includes the polyacrylate thickener, more preferably, the acrylates/C10-30 alkyl acrylate crosspolymer, even more preferably CARBOPOL® ETD 2020, wherein the thickening agents are present in an amount of from about 0.01 wt % to about 20 wt %, preferably, about 0.05 wt % to about 15 wt %, more preferably about 0.05 wt % to about 10 wt %, even more preferably about 0.1 wt % to about 2 wt %, or about 1 wt %, based on the total weight of the composition.

As used herein, the term or expression “humectant” may refer to a substance having affinity for water with stabilizing action on the water content of a material. The one or more humectants may include polyols, such as edible polyhydric alcohols (e.g., sugar alcohols). Illustrative humectants may be or include, but are not limited to, glycerin, butylene glycol, propylene glycol, sorbitol, xylitol, polyethylene glycol, or the like, or a combination thereof. In a preferred implementation, the humectants of the oral care composition include xylitol, glycerin, sorbitol, or a combination thereof. For example, the humectants include a combination of xylitol and glycerin.

The one or more humectants may be present in an amount of from 5 weight % to about 80 weight %, based on the total weight of the composition. For example, any one or more of the humectants may be present in an amount of from about 5 weight %, about 15 weight %, about 25 weight %, or about 35 weight % to about 45 weight %, about 55 weight %, about 65 weight %, about 75 weight %, or about 80 weight %, based on the total weight of the composition. In another example, any one or more of the humectants may be present in an amount of from about 5 weight % to about 80 weight %, about 15 weight % to about 75 weight %, about 25 weight % to about 65 weight %, about 35 weight % to about 55 weight %, or about 35 weight % to about 45 weight %, or about 40 wt %, based on the total weight of the composition. In a preferred implementation, the humectants may be present in an amount of about 25 weight % to about 55 weight %, preferably about 30 weight % to about 50 weight %, more preferably about 35 weight % to about 45 weight %, or about 40 weight %, based on the total weight of the composition.

In at least one implementation, the humectants are selected from xylitol, glycerin, or combinations thereof. For example, the humectants may include a combination of xylitol and glycerin. The xylitol may be present in an amount of from about 1 wt % to about 5 wt %, about 2 wt % to about 4 wt %, or about 3 wt %, based on the total weight of the oral care composition. The glycerin may be present in an amount of from about 25 weight % to about 55 weight %, preferably about 30 weight % to about 50 weight %, more preferably about 35 weight % to about 45 weight %, or about 40 weight %, based on the total weight of the composition.

The one or more solvents of the orally acceptable vehicle may be or include any suitable solvent compatible with the remaining components of the oral care product or the oral care composition thereof. Illustrative solvents of the orally acceptable vehicle may be or include, but are not limited to, water, such as purified water or deionized water, ethanol, or the like, or a combination thereof. In a preferred implementation, the orally acceptable vehicle includes water, more preferably demineralized water. Water may make up the balance of the oral care composition or the orally acceptable vehicle thereof. While water may make up the balance of the oral care composition or the orally acceptable vehicle thereof, water may be maintained or present in an amount of less than or equal to 65 wt %, less than or equal to 60 wt %, less than or equal to 55 wt %, less than or equal to 50 wt %, or less than or equal to 45 wt %, based on the total weight of the oral care composition. It should be appreciated that limiting the amount of water may reduce or prevent oxidation of one or more components of the oral care composition. For example, limiting the amount of water may reduce or prevent the oxidation of the one or more flavorants contained in the oral care composition, thereby improving the stability thereof.

The one or more pH modifying agents may be or include one or more bases or basifying agents, one or more acids or acidifying agents, one or more buffers or buffering agents, or a combination thereof. The one or more pH modifying agents may be capable of or configured to provide the oral care product or the oral care composition thereof a pH of from 5 to 9, from 5 to 8, from 5 to 8, from 6 to 8, from 7 to 8, or about 7.5. Illustrative pH modifying agent are known in the art and may include, but are not limited to, carboxylic, phosphoric, and sulfonic acids, acid salts (e.g., monosodium citrate, disodium citrate, monosodium malate, etc.), alkali metal hydroxides, such as sodium hydroxide, carbonates, such as sodium carbonate, bicarbonates, sesquicarbonates, borates, silicates, phosphates (e.g., monosodium phosphate, trisodium phosphate, pyrophosphate salts, etc.), or the like, or a combination thereof. It should be appreciated that any one or more of the pH modifying agents may be present in an amount effective to maintain the composition in an orally acceptable pH range. For example, the oral care composition may include sodium hydroxide in an amount effective to at least partially neutralize one or more acids, such as the one or more thickening agents.

The one or more flavorants or flavoring agents may be or include, but are not limited to, sweeteners, flavoring oils or essential oils, sucrose, sucralose, dextrose, polydextrose, dextrin, mannose, xylose, ribose, fructose, levulose, galactose, corn syrup, including high fructose corn syrup and corn syrup solids, partially hydrolyzed starch, lactose, maltose, xylitol, stevia, sodium cyclamate, perillartine, aspartame, liquorice, hydrogenated starch hydrolysate, sorbitol, mannitol, maltitol, isomalt, aspartame, neotame, saccharin and salts thereof (e.g., sodium saccharin), dipeptide-based intense sweeteners, cyclamates, dihydrochalcones saccharin or a salt thereof, or a combination thereof. Examples of the essential oils include oils of spearmint, peppermint, wintergreen, menthol, sassafras, clove, sage, eucalyptus, marjoram, cinnamon, lemon, lime, grapefruit, and orange. Any one or more of the flavorants or flavoring agents may be present in an amount of from greater than 0 wt % to less than or equal to 1 wt %, less than or equal to 0.8 wt %, less than or equal to 0.7 wt %, less than or equal to 0.6 wt %, less than or equal to 0.5 wt %, less than or equal to 0.4 wt %, less than or equal to 0.35 wt %, less than or equal to 0.30 wt %, less than or equal to 0.25 wt %, less than or equal to 0.20 wt %, less than or equal to 0.1 wt %, or less than or equal to 0.05 wt %.

As discussed above, the oral care composition may include one or more fluoride ion sources (e.g., soluble fluoride salts). A wide variety of fluoride ion-yielding materials may employed as sources of soluble fluoride. Examples of suitable fluoride ion-yielding materials may be found in U.S. Pat. No. 3,535,421 to Briner et al., U.S. Pat. No. 4,885,155 to Parran, Jr. et al., and U.S. Pat. No. 3,678,154 to Widder et al., the disclosures of which are incorporated herein by reference in their entirety to the extent they are consistent with the present disclosure. Illustrative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In a preferred implementation, the fluoride ion source includes sodium fluoride. The amount of the fluoride ion source present in the oral care composition may be less than 0.08 wt %. For example, the amount of the fluoride ion source present in the oral care composition may be less than 0.08 weight %, less than 0.07 weight %, less than 0.06 weight %, less than 0.05 weight %, or less than 0.04 weight %. In another implementation, the fluoride ion source is present in an amount to provide fluoride ions in a total amount of about 100 to about 20,000 ppm, about 200 to about 5,000 ppm, or about 500 to about 2,500 ppm.

The oral care product or the oral care composition thereof may include one or more surfactants. For example, the oral care composition may include one or more anionic surfactants, one or more cationic surfactants, one or more zwitterionic surfactants, one or more nonionic surfactants, or combinations thereof. Examples of suitable surfactants may be found in U.S. Pat. No. 3,959,458 to Agricola et al., U.S. Pat. No. 3,937,807 to Haefele, and U.S. Pat. No. 4,051,234 to Gieske et al., the disclosures of which are incorporated herein by reference in their entirety to the extent they are consistent with the present disclosure.

Illustrative anionic surfactants may include, but are not limited to, water-soluble salts of higher fatty acid monoglyceride monosulfates, such as a sodium salt of a monosulfated monoglyceride of hydrogenated coconut oil fatty acids, such as sodium N-methyl N-cocoyl taurate, sodium cocomonoglyceride sulfate. Illustrative anionic surfactants may also include higher alkyl sulfates. As used herein, “higher alkyl” refers to C₆₋₃₀ alkyl. For example, in a preferred implementation the anionic surfactant is sodium lauryl sulfate. The anionic surfactants may also include higher alkyl-ether sulfates. For example, the anionic surfactants may have a formula CH₃(CH₂)ₘCH₂(OCH₂CH₂)ₙOSO₃X, where m is 6-16, n is 1-6, and X is Na or K. In an exemplary implementation, m is 10, and n is 2, 3, or 4, and X is Na or K. For example, the anionic surfactant may be sodium laureth-2 sulfate (CH₃(CH₂)₁₀CH₂(OCH₂CH₂)₂OSO₃Na). In another implementation, the anionic surfactant may include higher alkyl aryl sulfonates, such as sodium dodecyl benzene sulfonate (sodium lauryl benzene sulfonate), and higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. In an exemplary implementation, the anionic surfactant is a water soluble salt of alkyl sulfates having from 10 to 18 carbon atoms in the alkyl radical and water soluble salts of sulfonated monoglycerides of fatty acids having from 10 to 18 carbon atoms. For example, the anionic surfactant may be or include, sodium lauryl sulfate, sodium lauroyl sarcosinate, sodium coconut monoglyceride sulfonates, or the like, and mixtures thereof.

The nonionic surfactant may function as an emulsifier. Illustrative nonionic surfactants may be or include, but are not limited to, poloxamers or the like. For example, the nonionic surfactants may include polysorbate 20, poloxamer 407, poloxamer 338, or the like, and mixtures thereof. The nonionic surfactants may also include, but are not limited to, ethoxylated and hydrogenated ethoxylated castor oils, such as those commonly designated as PEG NN castor oil or PEG NN hydrogenated castor oil, where “NN” designates the number of ethylene oxide units polymerized onto the castor oil to form the nonionic surfactant. For example, the nonionic surfactants may be or include PEG 16, 20, 25, 30, 40, 50, 60, 80, 100, 200, and combinations thereof. In a preferred implementation, the nonionic surfactant is polysorbate 20.

The amount of any one or more of the surfactants in the oral care product or the oral care composition thereof may be from about 0.010 wt %, about 0.020 wt %, about 0.030 wt %, about 0.040 wt %, about 0.045 wt %, about 0.049 wt %, or about 0.050 wt % to about 0.051 wt %, about 0.055 wt %, about 0.060 wt %, about 0.065 wt %, about 0.070 wt %, about 0.075 wt %, about 0.080 wt %, or greater. In another example, the amount of any one or more of the surfactants in the oral care product or the oral care composition thereof may be about 0.010 wt % to about 0.090 wt %, about 0.020 wt % to about 0.080 wt %, about 0.030 wt % to about 0.070 wt %, about 0.040 wt % to about 0.060 wt %, about 0.045 wt % to about 0.055 wt %, or about 0.050 wt % to about 0.051 wt %. In yet another example, the amount of any one or more of the surfactants in the oral care product or the oral care composition thereof may be greater than 0.010 wt %, greater than 0.020 wt %, greater than 0.030 wt %, greater than 0.040 wt %, greater than 0.045 wt %, greater than 0.049 wt %, or greater than 0.050 wt %. The amount of any one or more of the surfactants in the oral care product or the oral care composition thereof may also be from about 0.10 wt %, about 0.20 wt %, about 0.30 wt %, about 0.40 wt %, about 0.45 wt %, about 0.49 wt %, or about 0.50 wt % to about 0.51 wt %, about 0.55 wt %, about 0.60 wt %, about 0.65 wt %, about 0.70 wt %, about 0.75 wt %, about 0.80 wt %, or greater. In another example, the amount of any one or more of the surfactants in the oral care product or the oral care composition thereof may be about 0.10 wt % to about 0.90 wt %, about 0.20 wt % to about 0.80 wt %, about 0.30 wt % to about 0.70 wt %, about 0.40 wt % to about 0.60 wt %, about 0.45 wt % to about 0.55 wt %, or about 0.50 wt % to about 0.51 wt %. In yet another example, the amount of any one or more of the surfactants in the oral care product or the oral care composition thereof may be greater than 0.10 wt %, greater than 0.20 wt %, greater than 0.30 wt %, greater than 0.40 wt %, greater than 0.45 wt %, greater than 0.49 wt %, or greater than 0.50 wt %.

Illustrative antimicrobial agents or preservatives may be or include, but are not limited to, methylisothiazolinone (MIT), sodium benzoate, potassium sorbate, halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC), phenolic antiseptics, hexetidine, octenidine, sanguinarine, povidone iodine, delmopinol, salifluor, other metal ions (e.g., stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol, and other piperidino derivatives, nicin preparations, chlorite salts; and mixtures of any of the foregoing. In an exemplary implementation, the antibacterial agent includes cetylpyridinium chloride (CPC).

It should be appreciated by one having ordinary skill in the art, that the oral care products and/or the oral care composition thereof may include other additional ingredients/components. For example, the oral care products and/or the oral care composition thereof may include any one or more of the following: anti-caries agents, diluents, surface active agents or surfactants, mouth feel agents, sweetening agents, colorants or coloring agents, preservatives, antifoam agents (e.g., benzoic acid, sulfuric acid, glyceryl monostearate, etc.), or the like, or a combination thereof. It should further be appreciated by one having ordinary skill in the art that while general attributes of each of the above categories of materials may differ, there may be some common attributes and any given material may serve multiple purposes within two or more of such categories of materials. For example, clove oil may be capable of or configured to provide a flavor property as well as a therapeutic function.

The present disclosure may provide a method for preparing an oral care product or an oral care composition thereof. The method may include mixing, stirring, combining, or otherwise contacting an orally acceptable vehicle or carrier and the one or more phenolic compounds with one another. The method may include contacting the orally acceptable vehicle and the one or more phenolic compounds in respective amount to provide mouthwash.

The present disclosure may further provide methods for treating, preventing, or otherwise inhibiting one or more conditions of the oral cavity caused by or resulting from oxidative damage. Illustrative conditions of the oral cavity caused by or resulting from oxidative damage may be or include, but are not limited to, aging, gum disease, periodontal disease, or the like, or combinations thereof. The method may include contacting the oral cavity or a surface thereof with any one or more of the oral care compositions disclosed herein.

The method may also include decreasing and/or detecting (e.g., measuring or observing) a decrease in the amount of free radicals in the oral cavity and/or on surfaces thereof. The method may also include diagnosing or determining the presence of an oral condition caused by or resulting from oxidative damage.

The present disclosure may also provide methods for scavenging, reducing, reacting, preventing, or otherwise interacting with one or more free radicals and/or other reactive oxygen species in the oral cavity and/or on a surface thereof to thereby treat, prevent, or inhibit one or more conditions of the oral cavity caused by or resulting from oxidative damage. The method may include contacting the oral cavity or a surface thereof with any one or more of the oral care compositions disclosed herein. The method may also include decreasing and/or detecting a decrease in the amount of free radicals in the oral cavity and/or on the surfaces thereof. The method may also include diagnosing or determining the presence of an oral condition caused by or resulting from oxidative damage.

All ingredients for use in the compositions described herein should be orally acceptable. As used herein, “orally acceptable” may refer any ingredient that is present in a composition as described in an amount and form which does not render the composition unsafe for use in the oral cavity.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example

Seven oral care compositions (1)-(7) including varying amounts of a phenolic compound, namely, eugenol, were evaluated for their respective antioxidant efficacy. Oral care compositions (1)-(6) were prepared by preparing an orally acceptable carrier or vehicle and combining the orally acceptable vehicle with varying amounts of eugenol, as indicated in Table 1. Oral care composition (7), a negative control, was a commercially available oral care composition including no eugenol and green tea.

TABLE 1
Oral Care Composition

| | |
|---|---|
| 1 | Carrier + 0.0% Eugenol |
| 2 | Carrier + 0.005% Eugenol |
| 3 | Carrier + 0.008% Eugenol |
| 4 | Carrier + 0.01% Eugenol |
| 5 | Carrier + 0.015% Eugenol |
| 6 | Carrier + 0.025% Eugenol |
| 7 | Negative Control |

To evaluate the antioxidant efficacy, an ABTS (2,2′-azino-bis(3-ethylbenzothiazoline-6-sulphonic acid) assay (e.g., Trolox Equivalent Antioxidant Capacity Assay) was utilized in vitro. It should be appreciated that in the assay, ABTS is converted to its radical cation with the addition of an oxidant, such as sodium persulfate. The radical cation is blue-green in color and absorbs light at a wavelength of about 734 nm. Upon reaction of the radical cation with one or more antioxidants, the ABTS radical cation is converted back to its colorless neutral form, thereby fading from the blue-green color towards colorless or clear. The reaction was monitored spectrophotometrically. The results are summarized in Table 2. It should be appreciated that the lower the observed absorbance via the spectrophotometer, the lower the concentration of the free radicals; and thus, the relatively higher antioxidant efficacy, relatively higher free radical scavenging ability, and/or relatively higher antioxidant capacity of the oral care composition.

TABLE 2
Oral Care Compositions (1)-(6)

| | | |
|---|---|---|
| Oral Care Composition | Mean | Grouping¹ |

| | | | | |
|---|---|---|---|---|
| 1 | 0.4940 | A | | |
| 2 | 0.2210 | | B | |
| 3 | 0.0488 | | | C |
| 4 | 0.0292 | | | C |
| 5 | 0.0046 | | | C |
| 6 | 0.0036 | | | C |
| 7 | 0.5774 | A | | |

¹Means that do not share a letter are significantly different.

As indicated in Table 2, each of the oral care compositions (1)-(7) were denoted with a grouping by letters A, B, and C, using Tukey's range test at a 95% confidence level (CL). It should be appreciated that oral care compositions within the same group (i.e., A, B, C) are considered statistically or significantly the same with respect to antioxidant efficacy, free radical scavenging ability, and/or antioxidant capacity, while those of different groups are statistically or significantly different with respect to antioxidant efficacy, free radical scavenging ability, and/or antioxidant capacity.

As indicated by Table 2, the oral care composition (2) including about 0.005% eugenol provided significantly greater or higher antioxidant efficacy as compared to oral care composition (1), which did not include any eugenol. As further indicated in Table 2, it was surprisingly and unexpectedly discovered that oral care composition (3), which included about 0.008 wt % eugenol provided a statistically significant increase in the antioxidant activity as compared to oral care compositions (1) and (2). It was further surprisingly and unexpectedly discovered that a concentration of about 0.008 wt % was the lowest dosage that provided the best performance or best antioxidant activity, as increasing amounts of eugenol, as provided in oral care compositions (4)-(6) did not significantly increase the antioxidant efficacy or capacity or the respective oral care composition.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

1. An oral care composition, comprising an orally acceptable vehicle and one or more phenolic compounds, wherein the phenolic compounds are configured to provide or improve antioxidant efficacy of the oral care composition.

2. The oral care composition of claim 1, wherein the one or more phenolic compounds comprises one or more of hydroquinone, guaiacol, eugenol, gallic acid, any derivative thereof, or a combination thereof.

3. The oral care composition of claim 1, wherein the one or more phenolic compounds comprise eugenol or a derivative thereof.

4. The oral care composition of claim 1, wherein the one or more phenolic compounds comprise eugenol.

5. The oral care composition of claim 1, wherein the one or more phenolic compounds comprise a derivative of eugenol.

6. The oral care composition of claim 5, wherein the derivative of eugenol comprises one or more of eugenyl acetate, eugenyl formate, eugenyl benzoate, eugenol methyl ether, eugenol amyl ether, isoeugenol, dihydroeugenol, acetyl eugenol, methyleugenol, or combinations thereof.

7. The oral care composition of claim 1, wherein the one or more phenolic compounds are present in the oral care composition in an amount effective to treat, prevent, or inhibit one or more conditions of the oral cavity caused by or resulting from oxidative damage.

8. The oral care composition of claim 7, wherein the one or more phenolic compounds are present in an amount greater than or equal to 0.005%, based on the total weight of the oral care composition.

9. The oral care composition of claim 7, wherein the one or more phenolic compounds are present in an amount of from greater than or equal to about 0.005% to about 0.025%, based on the total weight of the oral care composition.

10. The oral care composition of claim 1, wherein the orally acceptable vehicle comprises one or more thickening agents, one or more humectants, one or more solvents, one or more pH modifying agents, one or more flavorants, one or more preservatives, one or more additional antioxidants, one or more sweeteners, one or more fluoride ion sources, one or more antimicrobial agent, one or more tartar control agents, or combinations thereof.

11. A method for preparing the oral care composition of claim 1, the method comprising contacting the orally acceptable vehicle and the one or more phenolic compounds with one another.

12. A method for treating, preventing, or inhibiting one or more conditions of an oral cavity resulting from oxidative damage, the method comprising contacting the oral care composition of claim 1 with the oral cavity.

13. A method for scavenging one or more free radicals in an oral cavity, the method comprising contacting the oral care composition of claim 1 with the oral cavity.

14. The method of claim 12, further comprising measuring a decrease in free radicals in the oral cavity.

15. The method of claim 12, further comprising diagnosing the presence of an oral condition resulting from oxidative damage in the oral cavity.

16. The method of claim 13, further comprising measuring a decrease in free radicals in the oral cavity.

17. The method of claim 13, further comprising diagnosing the presence of an oral condition resulting from oxidative damage in the oral cavity.

18. The oral care composition of claim 7, wherein the one or more phenolic compounds are present in an amount greater than or equal to 0.008%, based on the total weight of the oral care composition.

19. The oral care composition of claim 7, wherein the one or more phenolic compounds are present in an amount greater than or equal to 0.01%, based on the total weight of the oral care composition.

20. The oral care composition of claim 7, wherein the one or more phenolic compounds are present in an amount of from greater than or equal to about 0.008% to about 0.025%, based on the total weight of the oral care composition.
